(19) United States
(12) Patent Application Publication
(10) Pub. No.: US 2009/0163603 A1
(43) Pub. Date: Jun. 25, 2009

(54) SULFO-POLYMER POWDER AND SULFO-POLYMER POWDER BLENDS

(75) Inventor: Michael Fitzpatrick Wempe, Kingsport, TN (US)

Correspondence Address: BRETT L. NELSON;EASTMAN CHEMICAL COMPANY, 100 NORTH EASTMAN ROAD, KINGSPORT, TN, 37660-5075, US

(73) Assignee: Eastman Chemical Company, Kingsport, TN (US)

(21) Appl. No.: 12/263,010

(22) Filed: Oct. 31, 2008

Related U.S. Application Data
(60) Provisional application No. 61/015,356, filed on Dec. 20, 2007.

(51) Int. Cl. A61K 47/32; C08G 63/91

(52) U.S. Cl. 514/772.4; 525/418

(57) ABSTRACT

Sulfo-polymer powder and sulfo-polymer powder blends and subsequent preparation of dispersions of the sulfopolyester powder or sulfopolyester powder blends. The powders have reduced levels of volatiles and are generally whiter in color.

### CROSS-REFERENCE TO RELATED APPLICATIONS

This application claims priority to U.S. Provisional Application Ser. No. 61/015,356, filed Dec. 20, 2007 the disclosure of which is incorporated herein by reference in its entirety.

### BACKGROUND OF THE INVENTION

Sulfo-polyesters (such as Eastman AQ polymers) are high molecular weight amorphous polyesters that typically disperse directly in water without the need to incorporate organic co-solvents, surfactants, or amines. Sulfo-polyesters differ chiefly by their glass transition temperature (T_{g}), sometimes referred to as the ‘softening point’. The “dry” average T_{g} for each polyester may be denoted by its numerical designation. Examples include AQ 29, AQ38, AQ48, AQ55; hence, Eastman AQ 29D polymer has a T_{g} of ˜29.

There are two fundamental technical problems associated with these sulfopolyesters. Firstly, preparing dispersions of sulfopolyesters require hot water with significant mixing. Moreover, dispersions often have odor due to the presence of residual volatile components.

### BRIEF SUMMARY OF THE INVENTION

The present invention addresses the problems discussed above by enabling preparation of sulfo-polymer powder and sulfo-polymer powder blends and subsequent preparation of dispersions of said sulfopolyester powder or sulfopolyester powder blends, having reduced odor, with room temperature or lukewarm (i.e. water, water and alcohol mixtures, etc).

A first embodiment according to the present invention concerns a composition comprising at least one sulfonated copolyester powder.

Another embodiment concerns a method for producing a sulfonated copolyester powder comprising:

a) dispersing at least one sulfonated copolyester in a solvent to form a dispersion; and

b) drying said dispersion to form a powder.

Yet another embodiment concerns a cosmetic composition, comprising the composition described above and a cosmetically acceptable carrier.

### BRIEF DESCRIPTION OF THE DRAWINGS

The FIGURE shows a side-by-side comparison of an AQ polymer and the corresponding powder.

### DETAILED DESCRIPTION

The present invention concerns sulfopolyester powders and sulfopolyester blend powders and methods of making the powders as well as dispersions employing the powders.

As used throughout this application, the term “powder” shall mean particles in the range of 0.5-5000 μM. The science and technology of small particles is known as “micrometrics” (for a general review, see ‘Physical Pharmacy and Pharmaceutical Sciences’ Fifth Edition by Patrick J. Sinko, Lippincott Willams & Wilkins, 2006, ISBN: 0-7817-5027-X). The unit commonly used to describe particle size is the micrometer (μm). In general, optical microscopy may be used to measure particle-sizes of about 0.2 to about 100 μm; however, other techniques may also be used to determine approximate size ranges, such as sedimentation, coulter counter, air permeability, sieving, etc.

Techniques such as sieving, according to methods of the U.S. Pharmacopeia, may be used to determine ‘powder fineness’, or other properties of the corresponding powders and/or powder blends; for example, particle size and size distribution (i.e. average particle size, particle-size distribution (frequency distribution curve), number and weight distributions, particle number), particle volume, particle shape and surface area, pore size, porosity, particle density, bulkiness, flow properties, etc. Because many powders have a tendency to contain a non-symmetric particle size distribution, it is common to plot the log-normal distribution; commonly, this method results in a linear relationship. Subsequently, the “geometric mean diameter” (d_{g}; the particle size equivalent to 50% on the probability scale) may be obtained from plotting the logarithm of the particle size against the cumulative percent frequency on a probability scale. Therefore, as used throughout this application, powders shall be classified into different particle size ranges, such as: ‘extremely fine powders’ (i.e. dusty powders; 0.5-50 μm), ‘fine powders’ (50-100 μm), ‘coarse powders’ (100-1000 μm), and ‘granular powders’ (1000-5000 μm).

The sulfopolyester powders and/or sulfopolyester blend powders according to the present invention are generally prepared by techniques such as lyophilization, spray-drying, jet milling, spray freeze-drying, fluidized-bed spray coating, supercritical fluid methods, etc. The different techniques are based on different mechanisms of droplet/particle formation and/or drying (for a general review, see ‘Lyophilization of Biopharmaceuticals’ edited by Henry R. Costantino and Michael J. Pikal, AAPS Press, 2004, ISBN: 0-9711767-6-0).

Sulfo-polyester powder(s) and sulfo-polyester powder blend(s) are prepared by dispersing said sulfo-polyester or sulfo-polyester blend in a solvent or solvent mixture (e.g. water, water and alcohol mixtures, etc) at various temperatures. The starting sulfo-polyester or sulfo-polyester blend can be in pellet form, or, for example, can be a sample that has been chipped or ground up. The exact solvent, or solvent mixture, and temperatures used will be a function of the application end usage and/or required dispersion percentage.

Typical starting dispersions contain about 35% to about 2% by weight sulfo-polyester or sulfo-polyester blend, or between about 25% to about 5% weight sulfo-polyester or sulfo-polyester blend, or between about 15% to about 7% by weight sulfo-polyester or sulfo-polyester blend.

A non-exhaustive list of suitable solvents or solvent mixture includes: water, methanol, ethanol, propanol, isopropyl alcohol, polyethylene glycols, propylene glycols, etc., and solvent mixtures thereof. Typically the solvent/sulfo-polyester or sulfo-polyester blend mixture is dispersed at a temperature of about 90° C. to about 20° C., about 70° C. to about 30° C., or about 60° C. to about 40° C. Typically, the mixtures are stirred at an elevated temperature until the sulfo-polyester or sulfo-polyester blend is dispersed which can be a time period of about 72 h to about 0.1 h, of about 48 h to about 1 h, of about 24 h to about 12 h, or about 6 h to about 2 h.

The sulfo-polyester or sulfo-polyester blend powders can then be formed via, for example, lyophilization wherein the dispersion is first frozen and then lyophilized (i.e. freeze dried). Alternatively, the powders may be formed via other methods (spray-drying, jet milling, spray freeze-drying, fluidized-bed spray coating, supercritical fluid methods, etc.).

The resulting powders are readily dispersible and show a significant reduction in residual volatile components; hence the sulfo-polyester powders are purer materials. Furthermore, compared to the solid sulfo-polyester(s) on a weight to weight comparison, the corresponding solvent/sulfo-polyester powder or sulfo-polyester powder blends disperse at room temperature with rates which are statistically different from one another (for example, see the ‘Dispersion preparation from AQ powder example’); the powders have a much faster dissolution rate in a corresponding solvent, or solvent mixture, at room temperature. Various methods to evaluate dissolution are well known; for a general review, see ‘Physical Pharmacy and Pharmaceutical Sciences’ Fifth Edition by Patrick J. Sinko, Lippincott Willams & Wilkins, 2006, ISBN: 0-7817-5027-X. Therefore, one should readily appreciate that the application end usage and/or required dispersion percentage would dictate the exact solvent, or solvent mixture, and temperatures used to prepare dispersions from the powders. For example, a temperature of about 70° C. to about 10° C., about 50° C. to about 15° C., or about 35° C. to about 18° C. may be used. Typically, the mixtures are stirred at room temperature, or elevated temperature, until the sulfo-polyester powder or sulfo-polyester powder blend becomes dispersed which can be a time period of about 12 h to about 1 min, of about 6 h to about 5 min, or of about 2 h to about 15 min.

The resulting sulfo-polyester powder or sulfo-polyester blend powders according to the present invention can be used in, for example, sun care/skin care applications (i.e. creams, lotions and sprays), hairstyling applications (i.e. hair gel and hairspray), fragrant retention, color makeup and hairstyling applications, and adhesive removal such as re-pulping of paper and plastic and glass recycling.

### EXAMPLES

This invention can be further illustrated by the following examples, although it will be understood that these examples are included merely for purposes of illustration and are not intended to limit the scope of the invention unless otherwise specifically indicated.

### Materials

AQ29D, AQ38S, AQ48S, AQ55S, Eastek 1200 (aka AQ65D), AQ1045S, AQ1350S, AQ1950S, and EASTONE were obtained from Eastman Chemical Company. The “S” or “D” nomenclature used below refers to the solid or dispersed form of the polymer, respectively. Therefore, AQ29 and Eastek 1200 were obtained as dispersions; Eastek 1200 dispersion contains ˜2% propanol. HPLC grade water was purchased from Sigma-Aldrich (St. Louis, Mo., USA). A VWR SympHony SB20 pH meter was used (Oak Ridge, Tenn.). A Virtis (SP Industries Inc.; Warminster, Pa.) laboratory freeze dryer (model #4KBTXL-75) was used.

### Dispersion Preparation

Dispersions were prepared as follows:

### AQ38 Dispersion

AQ38 pellets (120 g) were weighed out into a beaker (100 mL). Water (280 mL) and a stir-bar were added to a beaker (500 mL). The water was heated. With continued heating, AQ38 was added (1.5 h) in portions with vigorous stirring. After an additional 30 min, the polymer appeared to be completely dispersed and was poured into a glass jar, allowed to slowly cool (6 h) to afford a milky white dispersion and capped. The next day, after mixing to ensure a homogenous dispersion, the pH at room temperature was taken (6.0±0.1). These methods afford an estimated 30% AQ38 dispersion.

### AQ48 Dispersion

AQ48 pellets (128 g) were weighed out into a beaker (250 mL). Water (428 mL) and a stir-bar were added to a beaker (1000 mL). The water was heated. With continued heating, AQ48 was added (1.5 h) in portions with vigorous stirring. After an additional 30 min, the polymer appeared to be completely dispersed and was poured into a glass jar, allowed to slowly cool (6 h) to afford a thick hazy light yellow, but translucent, dispersion and capped. The next day, after mixing to ensure a homogenous dispersion, the pH at room temperature was taken (5.6±0.1). These methods afford an estimated 23% AQ48 dispersion.

### AQ55 Dispersion

AQ55 pellets (120 g) were weighed out into a beaker (100 mL). Water (280 mL) and a stir-bar were added to a beaker (500 mL). The water was heated. With continued heating, AQ55 was added (1.5 h) in portions with vigorous stirring. After an additional 30 min, the polymer appeared to be completely dispersed and was poured into a glass jar, allowed to slowly cool (6 h) to afford a thick translucent dispersion and capped. The next day, after mixing to ensure a homogenous dispersion, the pH at room temperature was taken (6.2±0.1). These methods afford an estimated 30% AQ55 dispersion.

### AQ1045 Dispersion

AQ1045 block (yellow-orange sticky solid) was carefully cooled with liquid N₂ and pieces were carefully chipped off using a hammer and chisel. Afterwards, AQ1045 (120 g) was weighed out into a beaker (100 mL). Water (280 mL) and a stir-bar were added to a beaker (500 mL). The water was heated. With continued heating, AQ1045 was added (1.5 h) in portions with vigorous stirring. After an additional 30 min, the polymer appeared to be completely dispersed and was poured into a glass jar, allowed to slowly cool (6 h) to afford a milky white dispersion and capped. The next day, after mixing to ensure a homogenous dispersion, the pH at room temperature was taken (5.6±0.1). These methods afford an estimated 30% AQ1045 dispersion.

### AQ1350 Dispersion

AQ1 350 block (yellow-orange sticky solid) was carefully cooled with liquid N₂ and pieces were carefully chipped off using a hammer and chisel. Afterwards, AQ1 350 (85 g) were weighed out into a beaker (100 mL). Water (320 mL) and a stir-bar were added to a beaker (500 mL). The water was heated. With continued heating, AQ1350 was added (1.5 h) in portions with vigorous stirring. After an additional 30 min, the polymer appeared to be completely dispersed and was poured into a glass jar, allowed to slowly cool (6 h) to afford a milky white dispersion and capped. The next day, after mixing to ensure a homogenous dispersion, the pH at room temperature was taken (5.4±0.1). These methods afford an estimated 21% AQ1350 dispersion.

### AQ1950 Dispersion

AQ1950 block (yellow-orange sticky solid) was carefully cooled with liquid N₂ and pieces were carefully chipped off using a hammer and chisel. Afterwards, AQ1950 (85 g) was weighed out into a beaker (100 mL). Water (320 mL) and a stir-bar were added to a beaker (500 mL). The water was heated to nearly boiling. With continued heating, AQ1950 was added (1.5 h) in portions with vigorous stirring. After an additional 30 min, the polymer appeared to be completely dispersed and was poured into a glass jar, allowed to slowly cool (6 h) to afford a milky white dispersion and capped. The next day, after mixing to ensure a homogenous dispersion, the pH at room temperature was taken (5.7±0.1). These methods afford an estimated 21% AQ1950 dispersion.

### AQ2150 Dispersion

AQ2150 block (yellow-orange sticky solid) was carefully cooled with liquid N₂ and pieces were carefully chipped off using a hammer and chisel. Afterwards, AQ2150 (60 g) was weighed out into a beaker (100 mL). Water (336 mL) and a stir-bar were added to a beaker (500 mL). The water was heated. With continued heating, AQ2150 was added (1.5 h) in portions with vigorous stirring. After an additional 30 min, the polymer appeared to be completely dispersed and was poured into a glass jar, allowed to slowly cool (6 h) to afford a hazy light yellow, but translucent, dispersion and capped. The next day, after mixing to ensure a homogenous dispersion, the pH at room temperature was taken (5.5±0.1). These methods afford an estimated 15% AQ2150 dispersion.

### AQ2350 Dispersion

AQ2350 block (yellow-orange sticky solid) was carefully cooled with liquid N₂ and pieces were carefully chipped off using a hammer and chisel. Afterwards, AQ2350 (50 g) was weighed out into a beaker (100 mL). Water (243 mL) and a stir-bar were added to a beaker (500 mL). The water was heated. With continued heating, AQ2350 was added (1.5 h) in portions with vigorous stirring. After an additional 30 min, the polymer appeared to be completely dispersed and was poured into a glass jar, allowed to slowly cool (6 h) to afford a hazy light yellow, but translucent, dispersion and capped. The next day, after mixing to ensure a homogenous dispersion, the pH at room temperature was taken (5.5±0.1). These methods afford an estimated 19% AQ2350 dispersion.

### EASTONE Dispersion

EASTONE pellets (121 g) were weighed out into a beaker (100 mL). Water (350 mL) and a stir-bar were added to a beaker (500 mL). The water was heated. With continued heating, EASTONE was added (1.5 h) in portions with vigorous stirring. After an additional 30 min, the polymer appeared to be completely dispersed and was poured into a glass jar, allowed to slowly cool (6 h) to afford a milky white dispersion and capped. The next day, after mixing to ensure a homogenous dispersion, the pH at room temperature was taken (6.2±0.1). These methods afford estimated 25-26% EASTONE dispersion.

### AQ Powder Preparation: Dispersion Freeze Drying (Lyophilize)

Individually, the AQ29, AQ38, AQ48, AQ55, Eastek 1200 (AQ65), AQ1045, AQ1350, AQ1950, AQ2150, AQ2350, and EASTONE dispersions were mixed and samples poured into 250 mL round bottom flasks. In order to get the amount of added dispersion, the flasks were weighed before and after addition. The dispersions were then swirled and cooled with a dry-ice bath until frozen. Afterwards, the materials were lyophilized overnight to afford white to off-white powders; after removal, some of the powders became clear or were a sticky mass and presumed to be a function of retained water not removed, i.e. greater mass than the estimated polymer percentage.

The materials were then transferred out of the round bottoms into individual glass jars and capped. The results for the solids obtained after the samples were lyophilized afforded a way to measure the actual polymer dispersion percentage. Those results were as follows: AQ29 (29.7%), AQ38 (29.1%), AQ48 (22.5%), AQ55 (28.2%), Eastek 1200 (AQ65; 35.7%), AQ1045 (30.7%), AQ1350 (22.7%), AQ1950 (24.6%), AQ2150 (16.0%), AQ2350 (21.0%), and EASTONE (24.9%). Having these numbers in hand, dispersion blends were subsequently prepared by appropriate dispersion ratio mixing, freezing, and lyophilized. Examples of the various polymer powder and/or blends are summarized in Table 1. Furthermore, it was not until one approached around 25% of a sticky AQ polymer (i.e. AQ1045, AQ1350, AQ1950, AQ2150, AQ2350) before a powder blend became difficult to handle.

Dispersion Preparation from AQ Powder Example

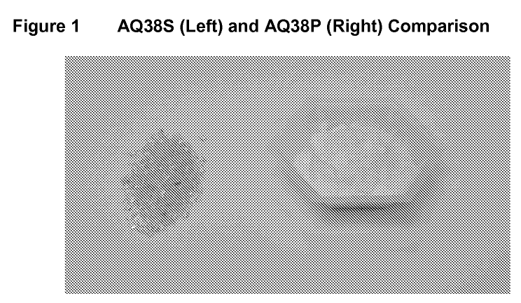

Using these polymer powder forms, one may readily prepare dispersions. The following example (˜7.3% dispersion preparation) has been included to illustrate the polymer powder utility. AQ38S pellets (3.0 g) and AQ38 powder (AQ38P; 3.0 g) were weighed out into separate beakers (50 mL) and both contained a stir-bar (FIG. 1). At the beginning of the preparation (time=0), and at the same time, the stirrers were placed on the same stirring setting. Next, room temperature water (38 mL) was added to each of the beakers. After one minute and less than 5 min, the powder form was completely dispersed. Whereas the pellets at 5 min essentially had limited to no evidence of dispersion formation; in fact, it took a few hours before the pellets even began to swell (i.e. become hydrated) and after 24 hours this mixture was still not a complete dispersion and much thicker than the polymer powder dispersion.

### CONCLUSION

Polymer dispersions were prepared but required warm/hot water with vigorous mixing. In general, a small batch of dispersion (100 grams with 20-30% AQ content) took 2-4 hours to prepare and subsequently required additional time to cool to room temperature. If room temperature water was used, it took 1 to 2 days to prepare the dispersion at which point the dispersion was still thicker than via the hot water method. After dispersions and dispersion blends were prepared, samples were frozen and lyophilized to afford white solids; the majority of these white solids were not tacky, easy to handle, and readily soluble in room temperature water.

In addition to the ability that one may readily prepare novel AQ powder blends, which may be used to readily prepare aqueous dispersions, this methodology significantly removed residual volatile components that were clearly present (i.e. there are sweet smelling volatiles present) in the original dispersions. The ability to offer powder sulfopolyesters and/or powder sulfoployester blends may offer users the advantage of easily and quickly preparing their dispersions with room temperature or lukewarm or hot water and/or water/organic solvent systems.

TABLE 1
AQ Polymer and Polymer Blend Summary

| | Polymer/Polymer Blend | Appearance |
|---|---|---|
| 1 | AQ29 | White Powder |
| 2 | AQ38 | White Powder |
| 3 | AQ48 | White Powder |
| 4 | AQ55 | White Powder |
| 5 | AQ1045 | Clear Sticky Solid |
| 6 | AQ1350 | White Powder but Hygroscopic |
| 7 | AQ1950 | White Powder but Hygroscopic |
| 8 | EASTONE | White Powder |
| 9 | AQ29:AQ1045 (5:1 Blend) | White Powder |
| 10 | AQ29:AQ1045 (4:1 Blend) | White Powder |
| 11 | AQ29:AQ1350 (5:1 Blend) | White Powder |
| 12 | AQ29:AQ1950 (5:1 Blend) | White Powder |
| 13 | AQ29:EASTONE (1:1 Blend) | White Powder |
| 14 | AQ38:AQ1045 (5:1 Blend) | White Powder |
| 15 | AQ38:AQ1045 (4:1 Blend) | White Powder |
| 16 | AQ38:AQ1045 (3:1 Blend) | White Powder but Hygroscopic |
| 17 | AQ38:AQ1350 (5:1 Blend) | White Powder |
| 18 | AQ38:AQ1950 (5:1 Blend) | White Powder |
| 19 | AQ48:AQ1045 (5:1 Blend) | White Powder |
| 20 | AQ48:AQ1045 (4:1 Blend) | White Powder |
| 21 | AQ48:AQ1350 (5:1 Blend) | White Powder |
| 22 | AQ48:AQ1950 (5:1 Blend) | White Powder |
| 23 | AQ55:AQ1045 (5:1 Blend) | White Powder |
| 24 | AQ55:AQ1045 (4:1 Blend) | White Powder |
| 25 | AQ55:AQ1350 (5:1 Blend) | White Powder |
| 26 | AQ55:AQ1950 (5:1 Blend) | White Powder |
| 27 | EASTONE:AQ1045 (5:1 Blend) | White Powder |
| 28 | EASTONE:AQ1045 (4:1 Blend) | White Powder |
| 29 | EASTONE:AQ1350 (5:1 Blend) | White Powder |
| 30 | EASTONE:AQ1950 (5:1 Blend) | White Powder |
| 31 | AQ38:AQ55:AQ1045 | White Powder |
| | (2:2:1 Blend) | |

The invention has been described in detail with particular reference to preferred embodiments thereof, but it will be understood that variations and modifications can be effected within the spirit and scope of the invention.

We claim:

1. A composition comprising at least one sulfonated copolyester powder.

2. The composition according to claim 1, wherein said sulfonated copolyester has a molecular weight of between about 5,000 and about 30,000.

3. The composition according to claim 2, wherein said sulfonated copolyester has a molecular weight of between about 7,000 and about 20,000.

4. The composition according to claim 3, wherein said sulfonated copolyester has a molecular weight of between about 10,000 and about 15,000.

5. The composition according to claim 1, wherein said sulfonated copolyester has a glass transition temperature of between about 10° C. and about 90° C.

6. The composition according to claim 5, wherein said sulfonated copolyester has a glass transition temperature of between about 10° C. and about 40° C.

7. The composition according to claim 6, wherein said sulfonated copolyester has a glass transition temperature of between about 70° C. and about 40° C.

8. The composition according to claim 6, wherein said sulfonated copolyester has a transition temperature of between about 90° C. and about 70° C.

9. The composition according to claim 1, wherein said composition is substantially free of residual volatile components.

10. A method for producing sulfonated copolyester powder comprising:
a) dispersing at least one sulfonated copolyester in a solvent to form a dispersion; and
b) drying said dispersion to form a powder.

11. The method according to claim 10, further comprising freezing said dispersion prior to drying.

12. The method according to claim 10, wherein said drying is by lyophilization, spray-drying, jet milling, spray freeze-drying, fluidized-bed spray coating, or supercritical fluid methods.

13. A cosmetic composition, comprising the composition according to claim 1 and a cosmetically acceptable carrier.
